(19) United States
(12) Patent Application Publication
(10) Pub. No.: US 2005/0266066 A1
(43) Pub. Date: Dec. 1, 2005

(54) Phospholipid membrane preparation

(75) Inventors: Tetsuya Uchida, Saitama-shi (JP); Masato Mori, Ota-ku (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignees: NOF Corporation, Tokyo (JP); National Institute of Infectious Diseases, Tokyo (JP)

(21) Appl. No.: 10/969,543

(22) Filed: Oct. 20, 2004

(30) Foreign Application Priority Data
Oct. 20, 2003 (JP) 360047/2003

(51) Int. Cl. A61K 9/127

(52) U.S. Cl. 424/450

(57) ABSTRACT

The present invention aims at providing a phospholipid membrane preparation wherein an antigen or an allergen is bound onto the surface of a phospholipid membrane comprising a phospholipid containing an acyl group or a hydrocarbon group having 10 to 12 carbon atoms, and a stabilizer of a phospholipid membrane. The present invention provides a phospholipid membrane preparation having an immune response controlling function that suppresses production of IgE antibody to increases production of practically sufficient IgG antibody and usable as a vaccine that does not easily cause an allergic response.

### TECHNICAL FIELD OF THE INVENTION

The present invention relates to a phospholipid membrane preparation wherein an antigen or an allergen is bound onto the surface of a phospholipid membrane comprising a phospholipid containing an acyl group or a hydrocarbon group having 10 to 12 carbon atoms, and a stabilizer of a phospholipid membrane. More particularly, the present invention relates to a phospholipid membrane and a phospholipid composition, which are the starting materials thereof.

### BACKGROUND OF THE INVENTION

As a substance causing what is called allergic conditions posing problems in humans, livestock and animals such as pets and the like, those mainly present as environmental factors such as acarian antigen, ragweed antigen, orchard grass antigen, cedar pollen antigen and the like, and allergens present in food such as egg, milk, buckwheat, peanut, fish, shellfish and the like are known. The patients and animal patients with these allergies encounter extreme difficulty in intentionally avoiding exposure to allergens, and strongly desire provision of effective treatment or prophylactic medicine. The onset of allergy is considered to be attributable to the production of an IgE antibody which is one of the antibodies. Therefore, suppression of the production of IgE antibody, or suppression of the production of IgE antibody combined with enhancement of the production of IgG antibody, is considered to enable treatment or improvement of allergic conditions.

As one technique aiming at treatment-improvement of allergic diseases, a hyposensitization therapy is known. In the hyposensitization therapy, a small amount of an aqueous solution of allergen is administered repeatedly within the range where the allergic conditions are tolerable, and regression of immune responses (IgE antibody production) is induced. There are a number of problems in achieving a certain beneficial effect by a hyposensitization therapy, such as individual difference in immune responses, the level of allergic conditions of patients or animal patients, necessity of a long-term treatment and the like. In addition, since the hyposensitization therapy is a passive treatment method aiming at induction of the regression of immune responses, many problems arise for this method to widely prevail as an effective treatment method.

As an improvement of this hyposensitization therapy, a treatment method is known, which comprises including an allergen in a particular liposome, orally administering or subcutaneously injecting the liposome to allergic patients or animal patients to lead the allergic conditions of the patients or animal patients to regression. However, since this treatment method also aims at making a hyposensitization therapy a safer, more effective and more comfortable method, it remains unchanged that this method is a passive treatment method aiming at induction of the regression of immune responses, which the hyposensitization therapy is based on. In other words, it does not possess an active function to control immunocompetence such as positive encouragement of the balance of production between IgG antibody and IgE antibody.

Moreover, as a method using a liposome for the treatment or improvement, a method using a known reactive phospholipid to allow an allergen to bind to a liposome is also known. While these techniques aim at effects of suppressing production of IgE antibody and enhancing production of IgG antibody, they fail to show performance meeting practical use because they have insufficient immunoregulating ability and require increased dose or higher administration frequency and the like.

These means of treating or improving allergic conditions are either a passive technique (hyposensitization therapy) that gradually decreases or regresses reactivity with allergen, or a technique that aims at effects of suppressing production of IgE antibody and enhancing production of IgG antibody, but fails to show sufficient effects. Therefore, they require intermittent and frequent administrations or administration in large amounts, and are still insufficient to make the practice of treatment widely prevail. Thus, the development of a preparation for the treatment or improvement of allergy, which provides sufficiently high practical effectiveness and which can be used safely, has been demanded.

In humans, livestock, animals such as pets and the like, and fish, moreover, various infections, whose pathogen is virus, bacteria etc., are known as diseases. For the prophylaxis of these infectious diseases, vaccines are widely used. As such vaccines, an aluminum hydroxide mixed vaccine wherein aluminum hydroxide has been added to an antigen solution to insolubilize the antigen, or, what is called a precipitated vaccine, is frequently used. To be specific, adsorbed diphtheria toxoid, absorbed diphtheria-tetanus combined toxoid, absorbed tetanus toxoid, adsorbed habu venom toxoid, absorbed hepatitis B vaccine, absorbed purified pertussis vaccine, absorbed diphtheria-purified pertussis-tetanus combined vaccine and the like have been used in practice.

In general, when a vaccine is inoculated, a living organism acquires immunocompetence and shows resistance by the production of IgG antibody or cell immunity. When a vaccine is inoculated, however, an unpreferable immune response sometimes occurs in living organisms, and the prime example is allergic response (side effect) upon vaccine inoculation. Such allergic responses are mostly caused by excessive production of IgE antibody and accompany red spots and swelling at vaccine inoculation sites, systemic shock and the like, where serious symptoms may possibly risk life. As mentioned above, besides the main immune responses such as production of IgG antibody that leads to the prophylaxis of infectious diseases and the like, occurrence of allergic response as an immune response not aimed at raises a big problem for vaccine inoculation.

As a main causative substance of allergic responses, the antigen itself used for vaccine, components contained in the antigen (e.g., protein in virus culture broth etc.) or adjuvant components used for the aforementioned precipitated vaccine, such as aluminum hydroxide gel and the like, and the like have been conventionally considered, but the causative substance has not been sufficiently identified yet. Therefore, techniques to suppress IgE antibody production that causes allergic responses have been variously and strenuously studied. However, a technique affording sufficient effect in practice has not been established yet.

In recent years, in the context of the prophylaxis of such infectious diseases, the following techniques have been disclosed. It is a technique working on the enhancement of IgG antibody production and suppression of IgE antibody production by binding an antigen to a liposome surface using a known reactive phospholipid (JP-A-09-012480). However, this technique shows markedly low and insufficient immunoregulating performance and is associated with many problems in practice, such as increased dose, increased frequency of administration and the like. That is, when a vaccine put into practice, which uses aluminum hydroxide gel as an adjuvant, and the above-mentioned liposome are compared in terms of immunity induction performance, with the production amount of IgG antibody using an equivalent amount of antigen as an index, the above-mentioned liposome shows about 1/10 lower effect than does the vaccine. Therefore, for the above-mentioned liposome to achieve an effect equivalent to that of the vaccine, a 10-fold amount of injection becomes necessary, or 10 times more frequent administration is considered to be necessary, which poses many practical problems.

In addition, a liposome with an antigen bound to its surface is known, wherein a special hydrophobic peptide bound to a physiologically active substance or antigen is inserted into a liposome membrane (WO02/098465). In this liposome, various physiologically active substances are bound to the surface of the liposome, and in an example close to the application to a vaccine, a production enhancing effect on γ-INF known as a cytokine that increases immunocompetence is mentioned. However, the above-mentioned reference does not specifically disclose that a technique to bind a physiologically active substance to the surface of a liposome using such a special peptide is applicable as a vaccine. In addition, the above-mentioned reference does not at all consider suppression of IgE antibody production and a practically sufficient IgG antibody production-enhancing effect, and does not indicate any specific solving means. Moreover, this technique has a substantial risk of a particular peptide being recognized as a foreign substance by the living organism, which in turn induces production of IgE antibody, and inducing a new allergic response. In addition, since the peptide does not get along well with acyl group and cholesterol that provide a hydrophobic environment in a phospholipid membrane, such as liposome and the like, it is feared that the stability of the preparation may be impaired or a peptide bound to a physiologically active substance may be released from the membrane, thus varying the effectiveness on the living organisms.

Furthermore, an antigen-bound liposome, wherein a hydrophobic peptide bound to an antigen is inserted into a liposome membrane, has been known (JP-T-2002-526436). In this technique, a liposome comprising a phospholipid, wherein the fatty acid has 14 to 18 carbon atoms, and cholesterol in a proportion of not more than 10% is used and a peptide bound to antigen is inserted into a liposome membrane, whereby an antigen-bound liposome is obtained. Moreover, the survival rate of test animals was measured as an immunological test using such liposome, and the effect of the vaccine was evaluated. Therefore, this liposome does not aim at provision of a vaccine that does not easily cause an allergic response. In this technique, since a hydrophobic domain of the peptide has 15-500 amino acids and a phospholipid wherein the fatty acid has 14 to 18 carbon atoms is used, these may become instable in the liposome membrane. Thus, the above-mentioned technique is associated with many problems, such as a risk of inducing an allergic response, lack of stability as a preparation, and further, high possibility of inconsistent effectiveness in living organisms and the like.

None of the aforementioned prescriptions using a liposome comprising an antigen or a physiologically active substance bound to the surface thereof discloses a vaccine that does not easily cause an allergic response. To be specific, they have a practically sufficient IgG antibody production-enhancing ability, do not disclose an IgE antibody production-suppressing effect, require intermittent and frequent administration or administration in large amounts to achieve a sufficient infection preventive effect, and are still insufficient techniques to make the practice of treatment widely prevail. Thus, the development of a vaccine preparation free of allergic response, which shows sufficiently high practical effectiveness and which can be used safely, has been demanded.

### SUMMARY OF THE INVENTION

The present invention has been made in view of such actual situation, and its problem to be solved is provision of a phospholipid membrane preparation having an immune response controlling function that suppresses production of IgE antibody to increases practically sufficient production of IgG antibody, and usable as a vaccine that does not easily cause an allergic response.

The present inventors have conducted intensive studies in an attempt to solve the above-mentioned problems and found that the above-mentioned problems can be solved by affording a phospholipid membrane preparation comprising a phospholipid membrane comprising a particular phospholipid and a stabilizer, wherein an antigen or an allergen is bound to the surface of the membrane, which resulted in the completion of the present invention.

Accordingly, the present invention provides the following.

(1) A phospholipid membrane preparation comprising a phospholipid membrane comprising a phospholipid having an acyl group or a hydrocarbon group having 10 to 12 carbon atoms and a stabilizer of a phospholipid membrane, wherein an antigen or an allergen is bound to the surface of the membrane.

(2) The phospholipid membrane preparation of the above-mentioned (1), wherein the above-mentioned phospholipid is at least one kind selected from diacylphosphatidylserine, diacylphosphatidylglycerol, diacylphosphatidic acid, diacylphosphatidylinositol, diacylphosphatidylcholine, diacylphosphatidylethanolamine, succinimidyl-diacylphosphatidylethanolamine and maleimide-diacylphosphatidylethanolamine.

(3) The phospholipid membrane preparation of the above-mentioned (1), wherein the above-mentioned phospholipid comprises at least one selected from diacylphosphatidylserine, diacylphosphatidylglycerol and diacylphosphatidic acid.

(4) The phospholipid membrane preparation of the above-mentioned (1), which comprises a lipid wherein an antigen or an allergen is bound by an ionic bond or a covalent bond.

(5) The phospholipid membrane preparation of any of the above-mentioned (1) to (4), wherein the above-mentioned stabilizer is cholesterol.

(6) The phospholipid membrane preparation of any of the above-mentioned (1) to (5), wherein the phospholipid membrane preparation is a liposome preparation.

(7) A liposome preparation comprising 1-99.6 mol % of an acidic phospholipid comprising an acyl group or a hydrocarbon group having 10 to 12 carbon atoms, 0.2-75 mol % of a stabilizer of a phospholipid membrane and 0.2-80 mol % of a lipid comprising an acyl group or a hydrocarbon group having 10 to 12 carbon atoms, which is bound to an antigen or an allergen.

(8) A liposome preparation comprising 1-99.5 mol % of an acidic phospholipid comprising an acyl group or a hydrocarbon group having 10 to 12 carbon atoms, 0.2-75 mol % of a stabilizer of a phospholipid membrane, 0.2-80 mol % of a lipid comprising an acyl group or a hydrocarbon group having 10 to 12 carbon atoms, which is bound to an antigen or an allergen and 0.1-70 mol % of a neutral phospholipid having an acyl group or a hydrocarbon group having 10 to 12 carbon atoms.

(9) A phospholipid membrane comprising a phospholipid having an acyl group or a hydrocarbon group having 10 to 12 carbon atoms, a reactive lipid and a stabilizer of a phospholipid membrane.

(10) A phospholipid membrane comprising 1-99.6 mol % of an acidic phospholipid comprising an acyl group or a hydrocarbon group having 10 to 12 carbon atoms, 0.2-75 mol % of a stabilizer of a phospholipid membrane and 0.2-80 mol % of a reactive lipid having an acyl group or a hydrocarbon group having 10 to 12 carbon atoms.

(11) A phospholipid membrane comprising 1-99.5 mol % of an acidic phospholipid comprising an acyl group or a hydrocarbon group having 10 to 12 carbon atoms, 0.2-75 mol % of a stabilizer of a phospholipid membrane, 0.2-80 mol % of a reactive lipid having an acyl group or a hydrocarbon group having 10 to 12 carbon atoms and 0.1-70 mol % of a neutral phospholipid having an acyl group or a hydrocarbon group having 10 to 12 carbon atoms.

According to the present invention, a phospholipid membrane preparation wherein an antigen or an allergen is bound to the surface of a phospholipid membrane comprising a phospholipid having an acyl group or a hydrocarbon group having 10 to 12 carbon atoms and a stabilizer of a phospholipid membrane can be provided. Such phospholipid membrane reparation has an immune response controlling function to suppress production of IgE antibody and sufficiently increase in practice the production of IgG antibody. Therefore, the phospholipid membrane preparation of the present invention can afford a superior effect in that allergic conditions do not occur easily during treatment-improvement of allergic diseases and vaccine inoculation for the prophylaxis of infectious diseases. In addition, the phospholipid membrane preparation of the present invention can provide a phospholipid membrane preparation containing an antigen at a low concentration because it shows markedly high effects of suppressing the production of IgE antibody and enhancing the production of IgG antibody. Thus, the phospholipid membrane preparation of the present invention is useful as a vaccine for the treatment-improvement of allergic diseases and the prophylaxis of infectious diseases.

### DETAILED DESCRIPTION OF THE INVENTION

The present invention is explained in more detail in the following.

The phospholipid membrane preparation of the present invention is used for vaccine and treatment of allergy, comprises a phospholipid having an acyl group or a hydrocarbon group having 10 to 12 carbon atoms and a stabilizer of a phospholipid membrane, wherein an antigen or an allergen is bound to the surface of the phospholipid membrane.

The phospholipid membrane used for the phospholipid membrane preparation of the present invention consists of an amphiphilic surfactant phospholipid, and has a structure wherein the phospholipid forms an interface with a polar group facing toward the aqueous phase side and a hydrophobic group faces the opposite side of the interface. The form of the phospholipid membrane includes a liposome, a phospholipid double membrane, a phospholipid micelle, a phospholipid emulsion and the like. As used herein, the liposome has a double membrane structure of phospholipid having a confined space. In addition, the phospholipid double membrane has a two-layer structure and its membrane structure is indefinite. The phospholipid micelle and phospholipid emulsion have a single membrane structure of phospholipid. Of these, liposome and phospholipid micelle are preferable from the aspects of practicality, easiness of preparation design, convenience of production and quality management, and the like and liposome is most preferable.

The allergen to be used in the phospholipid membrane preparation of the present invention is not particularly limited as long as it is an allergen (allergic antigen) causing an allergic disease. The subject that develops allergic conditions includes, for example, human; pets such as dog, cat, bird and the like; and livestock such as chicken, duck, pig, cow, sheep and the like. As the allergen, allergens of human and other animals can be mentioned. Specific examples of these allergens include house dust; acarian antigen, ragweed antigen, orchard grass antigen, cedar pollen antigen, mugwort antigen, Japanese hop antigen, lesser bullrush antigen and the like; grains such as rice, wheat, buckwheat and the like; foods such as milk, egg yolk, egg white and the like; epidermis such as dog hair, cat hair, feather and the like; fungi such as Candida, aspergillus and the like; and the like. Of these, acarian antigen, ragweed antigen, orchard grass antigen, cedar pollen antigen and egg white (OVA) antigen are preferably used from the aspects of the number of allergic cases, frequent intake from food and the like. The above-mentioned allergens may be used alone or in a combination of two or more kinds thereof.

The antigen for vaccine to be used for the phospholipid membrane preparation of the present invention may be any as long as it can be used as a vaccine antigen. Specifically, substances that can be antigens for human; pets such as dog, cat, bird and the like; and livestock such as chicken, duck, pig, cow, sheep and the like can be mentioned. As these antigens, for example, various toxoids such as tetanus, diphtheria, habu venom and the like; viruses such as influenza, polimyelitis, Japanese encephalitis, measles, epidemic parotiditis, epidemic roseola, hydrophobia, febris flava, chickenpox, hepatitis A, hepatitis B, hepatitis C, hemorrhagic fever with renal syndrome, dengue hemorrhagic fever, rotavirus infectious disease, parvovirus, coronavirus, distemper virus, leptospire, infectious bronchitis virus, contagious leukemia virus, AIDS and the like; cholera; viral disease autumn fever; BCG; malaria and the like can be mentioned. Of these antigens, human antigens and pet antigens that pose problems of allergic response during vaccine inoculation are preferable. Furthermore, antigens such as tetanus, diphtheria, Japanese encephalitis, polimyelitis, epidemic roseola, epidemic parotiditis in human; parvovirus in pets, corona virus, distemper virus, leptospire, infectious bronchitis virus, contagious leukemia virus and the like are more preferable. The above-mentioned antigen may be used alone or in a combination of two or more kinds thereof.

The antigens and allergens are proteins, peptides, saccharides and the like, and can be bound to the surface of a phospholipid membrane via a functional group they have. As such functional group, amino group, thiol group, carboxyl group, hydroxyl group, disulfide group, hydrophobic group consisting of hydrocarbon group having a methylene chain, and the like can be mentioned. Of these, amino group, thiol group, carboxyl group, hydroxyl group and disulfide group can bind an antigen or an allergen to the surface of a phospholipid membrane via a covalent bond, the amino group and the carboxyl group can bind via an ionic bond, the hydrophobic group can bind via a hydrophobic bond between hydrophobic groups. Since, in many cases, the antigen and the allergen are proteins or peptides containing a functional group in a high proportion, they allow convenient practical application. From such aspects, the functional group that an antigen or an allergen has is preferably amino group, carboxyl group or thiol group. When the antigen or allergen is saccharide, its functional group is preferably a hydroxyl group from the same aspects.

To bind the functional group that the antigen or allergen has to a phospholipid membrane, the phospholipid membrane desirably has a functional group such as amino group, succinimide group, maleimide group, thiol group, carboxyl group, hydroxyl group, disulfide group, hydrocarbon group consisting of a hydrophobic group having a methylene chain and the like. Since an antigen and an allergen are mostly protein or peptide, as the functional group that the corresponding phospholipid membrane has, amino group, succinimide group and maleimide group are preferable. The combination of the functional group that the antigen or allergen has and a functional group that a phospholipid membrane has may be any as long as it does not influence the effect of the present invention, with preference given to a combination of an amino group and an aldehyde group, an amino group and an amino group, an amino group and a succinimide group, and a thiol group and a maleimide group. For ionic bond and hydrophobic bond, the order of binding of antigen or an allergen to a phospholipid membrane is convenient and preferable for easy processing of the phospholipid membrane preparation. In addition, a covalent bond is preferable in terms of binding stability of antigen or an allergen to a phospholipid membrane surface and preservation stability when practicing the phospholipid membrane preparation. The phospholipid membrane preparation of the present invention is characterized in that an antigen or an allergen is bound to the surface of a phospholipid membrane. As a result, for example, even after administration to a living organism by injection in a practical stage, since an antigen or an allergen is stably bound to the surface of a phospholipid membrane, the effect of the present invention can be enhanced more. From these aspects, a covalent bond is preferable as a bond of an antigen or an allergen and a phospholipid membrane.

A phospholipid membrane to be used for the phospholipid membrane preparation of the present invention contains a phospholipid having an acyl group or a hydrocarbon group having 10 to 12 carbon atoms and a stabilizer of a phospholipid membrane. A phospholipid to be used for the present invention has an acyl group or a hydrocarbon group having 10 to 12 carbon atoms. As the acyl group having 10 to 12 carbon atoms, decanoyl group, undecanoyl group and dodecanoyl group can be mentioned. As the hydrocarbon group having 10 to 12 carbon atoms, decyl group, undecyl group and dodecyl group can be mentioned. As the phospholipid, glycerophospholipid can be used preferably. In this case, the acyl group and the hydrocarbon group bound to the 1-position or 2-position of the glycerine residue that a phospholipid has may be the same or different. From the aspect of industrial productivity, the 1-position or 2-position of the glycerine residue is preferably the same. In addition, as the phospholipid, a phospholipid having an acyl group having 10 to 12 carbon atoms is preferably used. In the present specification, the phospholipid is a concept including a salt thereof. As the salt, a pharmacologically acceptable salt is preferable. As such salt, a salt of phospholipid and an inorganic base, a phospholipid and an organic base such as ammonium and the like, and the like can be mentioned. As a salt with an inorganic base, alkali metal salts such as sodium, potassium, lithium and the like, alkaline earth metal salts such as calcium, magnesium and the like, and the like can be mentioned. Of these, alkali metal salts such as sodium salt and the like are preferable. One kind or a combination of two or more kinds of the salts of phospholipid can be used.

The present invention aims at achieving an immune response controlling function characterized by suppressing production of IgE antibody to increase practically sufficient production of IgG antibody. To increase production of practically sufficient IgG antibody, the phospholipid preferably has an acyl group having 10 to 12 carbon atoms. When the carbon atoms of the acyl group exceeds 12, sufficient production of IgG antibody becomes difficult to achieve. When the carbon atoms of the acyl group is less than 10, the hydrophobic binding force of the hydrophobic group of phospholipid becomes small and compatibility between phospholipid and a phospholipid membrane stabilizer such as cholesterol and the like becomes lower, which in turn degrades stability of a phospholipid membrane. As a phospholipid containing an acyl group having 10 to 12 carbon atoms, acidic phospholipid, neutral phospholipid, a reactive phospholipid having a functional group, which is capable of binding an antigen or an allergen to the surface and the like can be mentioned. The kind and proportion thereof are appropriately determined depending on various needs.

As the acidic phospholipid, a phospholipid having an acyl group or a hydrocarbon group having 10 to 12 carbon atoms, such as phosphatidylserine, phosphatidylglycerol, phosphatidic acid, phosphatidylinositol and the like can be used. From the aspects of industrial supply, the quality for use as a pharmaceutical product and the like, diacylphosphatidylserine, diacylphosphatidylglycerol, diacylphosphatidic acid and diacylphosphatidylinositol, each having an acyl group having 10 to 12 carbon atoms are preferable. In terms of sufficiently potentiating IgG antibody production in practice, industrial supply, quality for use as a pharmaceutical product and the like, which are aimed at by the present invention, diacylphosphatidylserine and diacylphosphatidylglycerol are particularly preferable, and in terms of sufficiently potentiating IgG antibody production, diacylphosphatidylserine is most preferable.

As the neutral phospholipid, for example, phosphatidylcholine containing an acyl group or a hydrocarbon group having 10 to 12 carbon atoms and the like can be mentioned. The kind and proportion of the neutral phospholipid that can be used in the present invention are appropriately determined and used within the range that affords suppression of the production of IgE antibody and practically sufficient, increased production of IgG antibody, that the present invention works on as problems. A neutral phospholipid has a higher function of stabilizing a phospholipid membrane, as compared to the lipid, to which acidic phospholipid, and antigen or an allergen are bound, and can improve the stability of the membrane. From such aspects, the phospholipid membrane preparation of the present invention preferably contains a neutral phospholipid. After ensuring the contents of the acidic phospholipid, a lipid bound to an antigen or an allergen and a stabilizer of a phospholipid membrane to be used for achieving the suppression of the production of IgE antibody and practically sufficient and increased production of IgG antibody that the present invention aims at, the amount of use of neutral phospholipid can be determined.

Since the phospholipid membrane preparation of the present invention contains a lipid to which an antigen or an allergen is bound, a phospholipid membrane preparation wherein an antigen or an allergen is bound to the surface of the phospholipid membrane can be obtained. To obtain such lipid to which an antigen or an allergen is bound, a reactive lipid having an acyl group or a hydrocarbon group having 10 to 12 carbon atoms can be used. As the reactive lipid having an acyl group or a hydrocarbon group having 10 to 12 carbon atoms, phospholipid having a reactive functional group, miono or diacylglyceride, fatty acid, cationic lipid and the like can be mentioned. The kind and proportion of the reactive lipid can be appropriately determined according to various demands. The acyl group or hydrocarbon group having more than 12 carbon atoms and less than 10 carbon atoms are not preferable for the same reasons as above.

In terms of stability in the phospholipid membrane, industrial supply, quality for use as a pharmaceutical product and the like, a reactive phospholipid containing an acyl group having 10 to 12 carbon atoms is preferably used as the reactive lipid. As the reactive phospholipid, diacylphosphatidylethanolamine and a terminal modified product can be mentioned. In addition, diacylphosphatidylglycerol, diacylphosphatidylserine, diacylphosphatidic acid, diacylphosphatidylinositol and terminal modified product of these can be also used. Considering industrial availability, convenience of a step of binding an antigen or an allergen, yield and the like, diacylphosphatidylethanolamine or a terminal modified product thereof is preferably used. As the “terminal modified product” here, for example, a compound wherein one of the terminals of a bifunctional reactive compound is bound to the amino group of diacylphosphatidylethanolamine can be mentioned.

Diacylphosphatidylethanolamine can be obtained by, for example, introducing ethanolamine into diacylphosphatidylcholine as a starting material by a base exchange reaction of choline using phospholipase D. To be specific, a solution of diacylphosphatidylcholine in chloroform and a solution of phospholipase D and ethanolamine in water are mixed at an appropriate ratio to give a crude reaction product. Then, the crude reaction product is purified by silica gel column using a mixed solvent of chloroform/methanol/water to give the object diacylphosphatidylethanolamine. The conditions for column purification such as solvent composition ratio and the like can be appropriately determined and put to practice.

As the bifunctional reactive compound, a compound wherein one of the terminals has aldehyde group or succinimide group capable of reacting with the amino group of diacylphosphatidylethanolamine can be mentioned. As a bifunctional reactive compound having an aldehyde group, glyoxal, glutaraldehyde, succindialdehyde, terephthaldehyde and the like can be specifically mentioned. Of these, glutaraldehyde is preferable. As a bifunctional reactive compound having a succinimide group, dithiobis(succinimidylpropionate), ethylene glycol-bis(succinimidylsuccinate), disuccinimidylsuccinate, disuccinimidylsuberate, disuccinimidylglutarate and the like can be specifically mentioned.

As a bifunctional reactive compound having a succinimide group on one terminal and a maleimide group on the other terminal, N-succinimidyl-4-(p-maleimidephenyl)butylate, sulfosuccinimidyl-4-(p-maleimidephenyl)butylate, N-succinimidyl-4-(p-maleimidephenyl)acetate, N-succinimidyl-4-(p-maleimidephenyl)propionate, succinimidyl-4-(N-maleimideethyl)-cyclohexane-1-carboxylate, sulfosuccinimidyl-4-(N-maleimideethyl)-cyclohexane-1-carboxylate, N-(γ-maleimidebutyryloxy)succinimide, N-(ε-maleimidecaproyloxy)succinimide and the like can be mentioned. Using these reactive compounds, a diacylphosphatidylethanolamine terminal modified product having a maleimide group is obtained. As mentioned above, by binding a functional group on one terminal of the bifunctional reactive compound to an amino group of diacylphosphatidylethanolamine, diacylphosphatidylethanolamine terminal modified product can be obtained.

As an example of the method of use of a reactive lipid, a phospholipid composition containing a reactive lipid is prepared, an antigen or an allergen is added and the mixture is subjected to a given treatment, whereby the phospholipid membrane preparation of the present invention wherein an antigen or an allergen is bound can be obtained. Alternatively, an antigen or an allergen is bound to a reactive lipid in advance, and this lipid is combined with a phospholipid and a stabilizer of a phospholipid membrane to give a phospholipid membrane, whereby the phospholipid membrane preparation of the present invention wherein an antigen or an allergen is bound.

In the present invention, as a stabilizer of a phospholipid membrane, sterols and tocopherols can be used. As the sterols, those generally known as sterols may be used, such as cholesterol, sitosterol, campesterol, stigmasterol, brassicasterol and the like. Of these, cholesterol is particularly preferable in view of availability and the like. As the tocopherols, those generally known as tocopherol may be used and, for example, commercially available α-tocopherol is preferable in view of availability and the like.

The phospholipid membrane preparation of the present invention preferably has the following composition.

A phospholipid membrane preparation comprising 1-99.6 mol % of a phospholipid having an acyl group or a hydrocarbon group having 10 to 12 carbon atoms, 0.2-75 mol % of a stabilizer of a phospholipid membrane, and 0.2-80 mol % of a lipid comprising an acyl group or a hydrocarbon group having 10 to 12 carbon atoms, which is bound to an antigen or an allergen.

The acidic phospholipid to be used in the present invention is added in a proportion of 1-99.6 mol % of the total constituent components of the phospholipid membrane. When the content is less than 1 mol %, the zeta potential becomes small, the stability of the phospholipid membrane becomes lower, and enhancement of the production of IgG antibody desired in the present invention cannot be sufficiently achieved with ease. When the content exceeds 99.6 mol %, the contents of a lipid to which an antigen or an allergen is bound and a stabilizer of a phospholipid membrane, necessary for achieving the object of the present invention, becomes insufficient. From such aspect, the content of acidic phospholipid is preferably 2-90 mol %, more preferably 4-80 mol %, most preferably 5-70 mol %.

The content of neutral phospholipid is 0-80 mol %, preferably 0.1-70 mol %, more preferably 0.1-60 mol %, most preferably 0.1-50 mol %, relative to the total constituent components of the phospholipid membrane. When it exceeds 80.0 mol %, an acidic phospholipid, a lipid to which an antigen or an allergen is bound and a stabilizer of a phospholipid membrane, necessary for achieving the object of the present invention, cannot be sufficiently used with ease.

A lipid to which an antigen or an allergen is bound to be used in the present invention can be added in a proportion of 0.2-80 mol % relative to the total constituent components of the phospholipid membrane. When it is less than 0.2 mol %, the amount of an antigen or an allergen bound to a phospholipid membrane, which is necessary for achieving the practically sufficient enhanced production of IgG antibody, becomes insufficient. When it exceeds 80 mol %, the stability of phospholipid membrane is degraded. The content of such lipid is preferably 1-55 mol %, more preferably 5-50 mol %.

The reactive lipid to be used in the present invention can be added in a proportion of 0.2-80 mol % relative to the total constituent components of the phospholipid membrane. When it is less than 0.2 mol %, a sufficient amount of an antigen or an allergen necessary for practically sufficient enhancement of the production of IgG antibody cannot be bound to a phospholipid membrane. When it exceeds 80 mol %, the stability of phospholipid membrane is degraded. From such aspects, the content of reactive lipid is preferably 1-55 mol %, more preferably 5-50 mol %.

A lipid bound to an antigen or an allergen, which is used in the present invention, is obtained by binding an antigen or an allergen to the aforementioned reactive lipid. When the reactive lipid is bound to an antigen or an allergen, the kind of the functional group to be used for binding, binding treatment conditions and the like can be appropriately determined as long as the effect of the present invention is not impaired. For example, When a terminal modified product of diacylphosphatidylethanolamine obtained by binding one terminal of disuccinimidylsuccinate, which is a bifunctional reactive compound, to a terminal amino group of diacylphosphatidylethanolamine is used as a reactive hospholipid, not less than 10%, preferably 10-99%, of the reactive phospholipids can be bound to an antigen or an allergen depending on the selection of various conditions such as binding treatment and the like. The reactive phospholipids that do not bind with an antigen or an allergen become an acidic phospholipid and are included in the phospholipid membrane preparation of the present invention.

The stabilizer of a phospholipid membrane usable in the present invention can be used in a proportion of 0.2-75 mol % of the total constituent components of the phospholipid membrane. In view of the practically sufficient, enhanced production of IgG antibody, which is an object of the present invention, a smaller content of the stabilizer is more preferable, but in view of the suppression of the production of IgE antibody, which is the other object of the present invention, not less than 0.2 mol % is preferable. When it exceeds 75 mol %, the stability of phospholipid membrane is impaired. The content of the stabilizer is preferably 1-70 mol %, more preferably 5-60 mol %, and still more preferably 10-55 mol %.

Preferable embodiments of the phospholipid membrane preparation of the present invention include the following compositions. The total of the compositions shown in the following is 100 mol %.

A phospholipid membrane preparation comprising 1-99.6 mol % of an acidic phospholipid comprising an acyl group or a hydrocarbon group having 10 to 12 carbon atoms, 0.2-75 mol % of a stabilizer of a phospholipid membrane, and 0.2-80 mol % of a lipid comprising an acyl group or a hydrocarbon group having 10 to 12 carbon atoms, which is bound to an antigen or an allergen.

A phospholipid membrane preparation comprising 2-90 mol % of an acidic phospholipid comprising an acyl group or a hydrocarbon group having 10 to 12 carbon atoms, 5-60 mol % of a stabilizer of a phospholipid membrane, and 1-55 mol % of a lipid comprising an acyl group or a hydrocarbon group having 10 to 12 carbon atoms, which is bound to an antigen or an allergen.

A phospholipid membrane preparation comprising 4-80 mol % of an acidic phospholipid comprising an acyl group or a hydrocarbon group having 10 to 12 carbon atoms, 10-55 mol % of a stabilizer of a phospholipid membrane, and 5-50 mol % of a lipid comprising an acyl group or a hydrocarbon group having 10 to 12 carbon atoms, which is bound to an antigen or an allergen.

When a phospholipid membrane preparation contains a neutral phospholipid, preferable embodiments include the following compositions.

A phospholipid membrane preparation comprising 1-99.6 mol % (preferably 1-99.5 mol %) of an acidic phospholipid comprising an acyl group or a hydrocarbon group having 10 to 12 carbon atoms, 0.2-75 mol % of a stabilizer of a phospholipid membrane, 0.2-80 mol % of a lipid comprising an acyl group or a hydrocarbon group having 10 to 12 carbon atoms, which is bound to an antigen or an allergen and 0.1-70 mol % of a neutral phospholipid.

A phospholipid membrane preparation comprising 2-90 mol % of an acidic phospholipid comprising an acyl group or a hydrocarbon group having 10 to 12 carbon atoms, 5-60 mol % of a stabilizer of a phospholipid membrane, 1-55 mol % of a lipid comprising an acyl group or a hydrocarbon group having 10 to 12 carbon atoms, which is bound to an antigen or an allergen and 1-60 mol % of a neutral phospholipid.

A phospholipid membrane preparation comprising 4-80 mol % of an acidic phospholipid comprising an acyl group or a hydrocarbon group having 10 to 12 carbon atoms, 10-55 mol % of a stabilizer of a phospholipid membrane, 5-50 mol % of a lipid comprising an acyl group or a hydrocarbon group having 10 to 12 carbon atoms, which is bound to an antigen or an allergen and 0.1-50 mol % of a neutral phospholipid.

When the acyl group or hydrocarbon group of acidic phospholipids has 10 carbon atoms, the following compositions are particularly preferably prepared.

A phospholipid membrane preparation comprising 5-70 mol % of an acidic phospholipid comprising an acyl group or a hydrocarbon group having 10 carbon atoms, 10-55 mol % of a stabilizer of a phospholipid membrane, 5-50 mol % of a lipid comprising an acyl group or a hydrocarbon group having 10 to 12 carbon atoms, which is bound to an antigen or an allergen and 0.1-50 mol % of a neutral phospholipid.

When the acyl group or hydrocarbon group of acidic phospholipid has 10 carbon atoms, the following compositions are more preferably prepared.

A phospholipid membrane preparation comprising 5-40 mol % of the total of diacylphosphatidylserine or diacylphosphatidylglycerol comprising an acyl group or a hydrocarbon group having 10 carbon atoms, 10-55 mol % of a stabilizer of a phospholipid membrane, 5-50 mol % of a lipid comprising an acyl group or a hydrocarbon group having 10 carbon atoms, which is bound to an antigen or an allergen and 0.1-50 mol % of a neutral phospholipid.

When the acyl group or hydrocarbon group of acidic phospholipid has 11 or 12 carbon atoms, the following compositions are preferably prepared.

A phospholipid membrane preparation comprising 5-70 mol % of an acidic phospholipid comprising an acyl group or a hydrocarbon group having 11 or 12 carbon atoms, 10-55 mol % of a stabilizer of a phospholipid membrane, 5-50 mol % of a lipid comprising an acyl group or a hydrocarbon group having 10 to 12 carbon atoms, which is bound to an antigen or an allergen and 0-50 mol % of a neutral phospholipid.

When the acyl group or hydrocarbon group of acidic phospholipid has 11 or 12 carbon atoms, the following compositions are more preferably prepared.

A phospholipid membrane preparation comprising 5-70 mol % of the total of diacylphosphatidylserine or diacylphosphatidylglycerol comprising an acyl group or a hydrocarbon group having 11 or 12 carbon atoms, 10-55 mol % of a stabilizer of a phospholipid membrane, 5-50 mol % of a lipid comprising an acyl group or a hydrocarbon group having 11 or 12 carbon atoms, which is bound to an antigen or an allergen and 0-50 mol % of a neutral phospholipid.

While the present invention is characterized in that an acyl group or a hydrocarbon group contained in a phospholipid and a lipid to which an antigen or an allergen is bound has 10 to 12 carbon atoms, a compound containing an acyl group or a hydrocarbon group having less than 10 or more than 12 carbon atoms may be contained as long as the effect of the present invention is not impaired. For example, the proportion of an acyl group or a hydrocarbon group having 10 to 12 carbon atoms is not less than 25 mol %, preferably not less than 50 mol %, more preferably not less than 75 mol %, still more preferably not less than 90 mol %, and most preferably not less than 97 mol %, relative to the total of all the acyl group or hydrocarbon group contained in the phospholipid membrane preparation of the present invention.

While the phospholipid membrane preparation of the present invention is characterized in that it contains a phospholipid an acyl group or a hydrocarbon group contained in a phospholipid and a lipid to which an antigen or an allergen is bound has 10 to 12 carbon atoms, a lipid other than phospholipid may be contained, as long as it has 10 to 12 carbon atoms, and the effect of the present invention is not impaired. The content of such lipid is, generally not more than 40 mol %, preferably not more than 20 mol %, more preferably not more than 10 mol %.

Moreover, as long as the effect of the present invention is not impaired, known phospholipid membrane constituent components capable of constituting a phospholipid membrane may be contained. A phospholipid membrane usable in the present invention can be obtained by a method comprising appropriately adding and processing a phospholipid, a reactive lipid, a stabilizer of a phospholipid membrane, an antigen or an allergen and the like as the constituent components, and adding these in a suitable solvent and the like. For example, when the phospholipid membrane is a liposome, it can be produced by methods including extrusion method, vortex mixer method, ultrasonication method, surfactant removal method, reverse-phase evaporation method, ethanol injection method, prevesicle method, French press method, W/O/W emulsion method, annealing method, freeze-thaw method and the like. When the phospholipid membrane is a phospholipid micelle, it can be produced by methods similar to those mentioned above.

In the present invention, the form of liposome is not particularly limited, liposomes having various sizes and shapes can be produced, such as a multilayer liposome, a small sheet of membrane liposome, a large sheet of membrane liposome and the like, by appropriately determining the aforementioned production methods of liposome. While the particle size of the liposome is not particularly limited in the present invention, it is preferably 20-600 nm, more preferably 30-500 nm, still more preferably 40-400 nm, particularly preferably 50-300 nm, and most preferably 70-230 nm, from the aspects of preservation stability and the like. The particle size refers to an average particle size, which can be measured by a dynamic light scattering method.

To improve physico-chemical stability of the liposome in the present invention, saccharides or polyhydric alcohols may be added to an inner aqueous phase and/or an outer aqueous phase of a liposome, during liposome preparation process or after preparation. Particularly, when a long-term preservation or preservation during preparation is necessary, saccharide or polyhydric alcohol is preferably added dissolved as a protecting agent of liposome, and moisture is removed by freeze drying to give a freeze dried product of a phospholipid composition.

As the saccharides, for example, monosaccharides such as glucose, galactose, mannose, fructose, inositol, ribose, xylose and the like; disaccharides such as saccharose, lactose, cellobiose, trehalose, maltose and the like; trisaccharides such as raffinose, melezitose and the like; oligosaccharide such as cyclodextrin and the like; polysaccharides such as dextrin and the like; sugar alcohols such as xylitol, sorbitol, mannitol, maltitol and the like; and the like can be mentioned. Of these saccharides, monosaccharides and disaccharides are preferable, and glucose and saccharose are more preferable from the aspect of availability.

As the polyhydric alcohols, for example, glycerol compounds such as glycerol, diglycerol, triglycerol, tetraglycerol, pentaglycerol, hexaglycerol, heptaglycerol, octaglycerol, nonaglycerol, decaglycerol, polyglycerol and the like; sugar alcohol compounds such as sorbitol, mannitol and the like; ethylene glycol, diethylene glycol, triethylene glycol, tetraethylene glycol, pentaethylene glycol, hexaethylene glycol, heptaethylene glycol, octaethylene glycol, nonaethylene glycol and the like can be mentioned. Of these, glycerol, diglycerol, triglycerol, sorbitol, mannitol, and polyethylene glycol having an average molecular weight of 400-10,000 are preferable from the aspect of availability. The concentration of the saccharides or polyhydric alcohols contained in an inner aqueous phase and/or an outer aqueous phase of a liposome is, for example, 1-20 wt %, preferably 2-10 wt %, of the total weight of the liposome suspension.

For preparation of the phospholipid membrane preparation of the present invention, a phospholipid membrane is prepared and then an antigen or an allergen is bound to the surface of the phospholipid membrane, whereby a phospholipid membrane preparation can be obtained conveniently. For example, a phospholipid membrane containing a phospholipid, a stabilizer of a phospholipid membrane, and a reactive lipid for binding an antigen or an allergen to the surface of a membrane, such as a liposome suspension is prepared, and about 2-10 wt % of sucrose, which is one of the aforementioned saccharides, is added to and dissolved in the outer aqueous phase. This saccharide added preparation is transferred to a 10 ml glass vial, placed in a lyophilizer, cooled to −40° C. and the like to freeze the sample, and a freeze dried product is obtained by a conventional method. Since the freeze dried product obtained here is free of water, a long-term preservation is possible. Where necessary, a particular antigen or an allergen is added and subjected to subsequent steps, whereby the final phospholipid membrane preparation of the present invention can be obtained conveniently and quickly. When interaction between an antigen or an allergen and a phospholipid membrane is strong and the stability is low and the like, the phospholipid membrane is preserved in the stage of a freeze dried product, and highly conveniently used where necessary after binding with an antigen or an allergen.

Preferable embodiments of the phospholipid membrane of the present invention prior to the reaction of an antigen or an allergen include the following. The total of the compositions shown in the following is 100 mol %.

A phospholipid membrane comprising 1-99.6 mol % of a phospholipid having an acyl group or a hydrocarbon group having 10 to 12 carbon atoms, 0.2-75 mol % of a stabilizer of a phospholipid membrane and 0.2-80 mol % of a reactive lipid having an acyl group or a hydrocarbon group having 10 to 12 carbon atoms.

Preferable embodiments of the phospholipid membrane include the following compositions.

A phospholipid membrane comprising 1-99.6 mol % of an acidic phospholipid comprising an acyl group or a hydrocarbon group having 10 to 12 carbon atoms, 0.2-75 mol % of a stabilizer of a phospholipid membrane, and 0.2-80 mol % of a reactive lipid comprising an acyl group or a hydrocarbon group having 10 to 12 carbon atoms.

A phospholipid membrane comprising 2-90 mol % of an acidic phospholipid comprising an acyl group or a hydrocarbon group having 10 to 12 carbon atoms, 5-60 mol % of a stabilizer of a phospholipid membrane, and 1-55 mol % of a reactive lipid comprising an acyl group or a hydrocarbon group having 10 to 12 carbon atoms.

A phospholipid membrane comprising 4-80 mol % of an acidic phospholipid comprising an acyl group or a hydrocarbon group having 10 to 12 carbon atoms, 10-55 mol % of a stabilizer of a phospholipid membrane, and 5-50 mol % of a reactive lipid comprising an acyl group or a hydrocarbon group having 10 to 12 carbon atoms.

When a neutral phospholipids is contained as a phospholipid membrane, preferable embodiments include the following compositions.

A phospholipid membrane comprising 1-99.6 mol % (preferably 1-99.5 mol %) of an acidic phospholipid comprising an acyl group or a hydrocarbon group having 10 to 12 carbon atoms, 0.2-75 mol % of a stabilizer of a phospholipid membrane, 0.2-80 mol % of a reactive lipid comprising an acyl group or a hydrocarbon group having 10 to 12 carbon atoms and 0.1-70 mol % of a neutral phospholipid.

A phospholipid membrane comprising 2-90 mol % of an acidic phospholipid comprising an acyl group or a hydrocarbon group having 10 to 12 carbon atoms, 5-60 mol % of a stabilizer of a phospholipid membrane, 1-55 mol % of a reactive lipid comprising an acyl group or a hydrocarbon group having 10 to 12 carbon atoms and 0.1-60 mol % of a neutral phospholipid.

A phospholipid membrane comprising 4-80 mol % of an acidic phospholipid comprising an acyl group or a hydrocarbon group having 10 to 12 carbon atoms, 10-55 mol % of a stabilizer of a phospholipid membrane, 5-50 mol % of a reactive lipid comprising an acyl group or a hydrocarbon group having 10 to 12 carbon atoms and 0.1-50 mol % of a neutral phospholipid.

When an acidic phospholipid having 10 carbon atoms is contained, the following composition is preferable.

A phospholipid membrane comprising 5-70 mol % of an acidic phospholipid comprising an acyl group or a hydrocarbon group having 10 carbon atoms, 10-55 mol % of a stabilizer of a phospholipid membrane, 5-50 mol % of a reactive lipid comprising an acyl group or a hydrocarbon group having 10 to 12 carbon atoms and 0.1-50 mol % of a neutral phospholipid.

When an acidic phospholipid having 10 carbon atoms is contained, the following composition is more preferable.

A phospholipid membrane preparation comprising 5-40 mol % of the total of diacylphosphatidylserine or diacylphosphatidylglycerol comprising an acyl group or a hydrocarbon group having 10 carbon atoms, 10-55 mol % of a stabilizer of a phospholipid membrane, 5-50 mol % of a reactive lipid comprising an acyl group or a hydrocarbon group having 10 to 12 carbon atoms and 0.1-50 mol % of a neutral phospholipid.

When an acidic phospholipid having 11 or 12 carbon atoms is contained, the following composition is preferable.

A phospholipid membrane comprising 5-70 mol % of an acidic phospholipid comprising an acyl group or a hydrocarbon group having 11 or 12 carbon atoms, 10-55 mol % of a stabilizer of a phospholipid membrane, 5-50 mol % of a reactive lipid comprising an acyl group or a hydrocarbon group having 10 to 12 carbon atoms and 0-50 mol % of a neutral phospholipid.

When an acidic phospholipid having 11 or 12 carbon atoms is contained, the following composition is more preferable.

A phospholipid membrane preparation comprising 5-70 mol % of the total of diacylphosphatidylserine or diacylphosphatidylglycerol comprising an acyl group or a hydrocarbon group having 11 or 12 carbon atoms, 10-55 mol % of a stabilizer of a phospholipid membrane, 5-50 mol % of a reactive lipid comprising an acyl group or a hydrocarbon group having 11 or 12 carbon atoms and 0-50 mol % of a neutral phospholipid.

The phospholipid membrane preparation of the present invention is characterized in that a lipid, to which an antigen or an allergen is bound, is contained. As a method to obtain a phospholipid membrane preparation containing a lipid, to which an antigen or an allergen is bound, a method comprising the following (A) or (B) can be mentioned.

(A) A method comprising preparing a phospholipid membrane containing a phospholipid, a reactive lipid and a stabilizer of a phospholipid membrane and adding an antigen or an allergen and a bifunctional reactive compound to allow binding of a functional group of a reactive lipid contained in the phospholipid membrane with a functional group of the antigen or allergen via the bifunctional reactive compound. As the bifunctional reactive compound, a compound used for preparing a terminal modified product of a reactive lipid can be used in the same manner. To be specific, as a bifunctional reactive compound having an aldehyde group, glyoxal, glutaraldehyde, succindialdehyde, terephthaldehyde and the like can be mentioned. Of these, glutaraldehyde is preferable. As the bifunctional reactive compound having a succinimide group, dithiobis(succinimidylpropionate), ethylene glycol-bis(succinimidylsuccinate), disuccinimidylsuccinate, disuccinimidylsuberate, disuccinimidylglutarate and the like can be used. Moreover, as the bifunctional reactive compound having a succinimide group on one terminal and a maleimide group on the other terminal, N-succinimidyl-4-(p-maleimidephenyl)butylate, sulfosuccinimidyl-4-(p-maleimidephenyl)butylate, N-succinimidyl-4-(p-maleimidephenyl)acetate, N-succinimidyl-4-(p-maleimidephenyl)propionate, succinimidyl-4-(N-maleimideethyl)-cyclohexane-1-carboxylate, sulfosuccinimidyl-4-(N-maleimideethyl)-cyclohexane-1-carboxylate, N-(γ-maleimidebutyryloxy)succinimide, N-(ε-maleimidecaproyloxy)succinimide and the like can be used. When the bifunctional reactive compound is used, a diacylphosphatidylethanolamine terminal modified product having a maleimide group as a functional group can be obtained.

(B) A method comprising preparing a phospholipid membrane containing a phospholipid, a reactive lipid and a stabilizer of a phospholipid membrane and adding an antigen or an allergen to allow binding of a functional group of a reactive lipid contained in the phospholipid membrane with a functional group of the antigen or allergen.

As the bond group in the above-mentioned (A) and (B), for example, ionic bond, hydrophobic bond and covalent bond can be mentioned. Moreover, specific examples of the covalent bond include schiff base bond, amide bond, thioether bond, ester bond and the like can be mentioned. In both of the above two methods, an antigen or an allergen can be bound to a reactive lipid contained in a phospholipid membrane. As a result, a lipid, to which an antigen or an allergen is bound, is formed in a phospholipid membrane.

In the above-mentioned method (A), a specific example of a method of binding a phospholipid membrane to be the starting material to an antigen or an allergen via a bifunctional reactive compound is the use of, for example, schiff base bond. As a method of binding a phospholipid membrane and an antigen or an allergen by a schiff base bond, for example, a method comprising preparation of a phospholipid membrane having an amino group on the surface, obtaining a suspension of the phospholipid membrane, adding an antigen or an allergen protein to the suspension of phospholipid membrane, and adding dialdehyde, which is a bifunctional reactive compound, to allow the amino group on the phospholipid membrane surface to bind to an amino group of the antigen or an allergen protein by a schiff base can be mentioned.

Specific examples of this binding step include the following methods.

(A-1) To obtain a phospholipid membrane having an amino group on the surface, diacylphosphatidylethanolamine having an acyl group or a hydrocarbon group having 10 to 12 carbon atoms is added to a starting material lipid of the phospholipid membrane to give a phospholipid membrane comprising a given amount of an amino group on the surface of the phospholipid membrane.

(A-2) An antigen or an allergen is added to the suspension of the phospholipid membrane.

(A-3) Then, glutaraldehyde is added as a bifunctional reactive compound, and the mixture is reacted for a given time to form a schiff base bond between the phospholipid membrane and the antigen or allergen.

(A-4) Thereafter, to inactivate excess glutaraldehyde, glycine as an amino group-containing water-soluble compound is added to the suspension of the phospholipid membrane to allow reaction.

(A-5) An antigen or an allergen not bound to the phospholipid membrane, reaction product of glutaraldehyde and glycine and excess glycine are removed by methods such as gel filtration, dialysis, ultrafiltration, centrifugal separation and the like to give a suspension of a phospholipid membrane comprising an antigen or an allergen bound to the surface.

As a specific example of the above-mentioned method (B), a method comprising introducing a reactive lipid having a functional group capable of forming a amide bond, thioether bond, schiff base bond, ester bond and the like into a phospholipid membrane can be mentioned. Specific examples of such functional group include succinimide group, maleimide group, amino group, imino group, carboxyl group, hydroxyl group, thiol group and the like. Examples of the reactive lipid to be introduced into a phospholipid membrane include a terminal modified product of an amino group terminal of diphosphatidylethanolamine containing an acyl group or a hydrocarbon group having 10 to 12 carbon atoms.

Specific examples of this binding step include the following methods.

(B-1) A diphosphatidylethanolamine containing an acyl group or a hydrocarbon group having 10 to 12 carbon atoms is reacted with one terminal of disuccinimidylsuccinate by a known method to give diphosphatidylethanolamine bound to disuccinimidylsuccinate having a succinimide group on one terminal.

(B-2) The disuccinimidylsuccinate bound diphosphatidylethanolamine is mixed with other constituent components of the phospholipid membrane by a known method to give a phospholipid membrane composition having a succinimide group on the surface.

(B-3) An antigen or an allergen protein is added to a suspension of the phospholipid membrane composition to allow reaction of an amino group of the antigen or allergen protein with a succinimide group on the surface of the phospholipid membrane.

(B-4) Unreacted antigen or allergen and reaction byproduct are removed by methods such as gel filtration, dialysis, ultrafiltration, centrifugal separation and the like to give a suspension of a phospholipid membrane comprising a lipid to which an antigen or an allergen is bound.

When a phospholipid membrane and an antigen or an allergen are bound, since antigen and allergen are mainly proteins, an amino group or a thiol group frequently contained as a reactive group is preferably used as a target in practice. When an amino group is the target, it is reacted with a succinimide group to form a schiff base bond. When a thiol group is the target, it is reacted with a maleimide group to form a thioether bond. Since the phospholipid membrane preparation of the present invention includes a phospholipid membrane preparation wherein an antigen or an allergen is bound to the surface, it functions for an allergy treatment or as a vaccine that prevents easy occurrence of allergic response. The liposome preparation of the present invention can be used for oral, transdermal, transmucosal, subcutaneous, intravenous, peritoneal administrations and the like.

In the following, formulations and method of use of the phospholipid membrane preparation of the present invention for the treatment of allergy are explained in detail.

As mentioned above, the phospholipid membrane preparation of the present invention obtained by methods such as gel filtration, dialysis, ultrafiltration, centrifugal separation and the like comprises an antigen or an allergen bound to the surface of a phospholipid membrane and is in a suspension state. As a solvent for suspending, aqueous solvents, such as distilled water; physiological saline; buffers (e.g., phosphate buffer, carbonate buffer, Tris buffer, acetate buffer and the like); and the like can be used. The pH of such aqueous solvent is 5-10, preferably 6-8. The aforementioned suspension of a phospholipid membrane can be powderized thereafter by treatments such as vacuum drying, freeze drying and the like. A phospholipid membrane in a powder can be preserved and dispersed in the above-mentioned aqueous solvent and the like when in use.

The administration of the phospholipid membrane preparation of the present invention can be started at any time point when allergic conditions are presented or before presenting allergic conditions. The level of immune response of the patients or animal patients against the allergen greatly varies depending on the life environment (environmental factor) inherently in contact with or family line (genetic factor) and the like. Therefore, the dose, administration period and administration frequency of the phospholipid membrane preparation of the present invention are preferably determined for each individual patient or animal patient. To be specific, a phospholipid membrane preparation is administered, IgE antibody titer against allergen in blood is followed and measured for some time to understand the progression of allergy treatment, and dose and administration period can be determined. The administration frequency can be, for example, every 1 week, 2 weeks, 3 weeks, 4 weeks, 2 months, 3 months, 4 months, 5 months, 6 months, 7 months, 8 months, 9 months, 10 months, 11 months or 12 months. In the case of patients and animal patients having an extremely sensitive allergy factor, it is preferable to continuously take the phospholipid membrane preparation of the present invention to improve allergic constitution.

The administration period of the phospholipid membrane preparation of the present invention is preferably a time point before being affected with an infectious disease and after vertical immunity from mother has become lower in animal patients. To be specific, 2-year-olds or after in human, 3-week-olds or after in dog and cat, 5-week-olds or after in pig and cow are preferable periods. When a novel and strongly toxic contagium evolves and the like, the phospholipid membrane preparation of the present invention is given for a necessary period of time to express function of the vaccine. The dose, administration period and administration frequency of the phospholipid membrane preparation vary depending on the similar environmental factors and genetic factors as mentioned above, and the level of immune response greatly varies depending on individual patients and animal patients. Therefore, they are preferably determined for each individual. To be specific, a phospholipid membrane preparation is administered, IgG antibody titer against antigen in blood is followed and measured for some time to understand the progression of vaccine treatment for prevention of infectious diseases, and dose and administration period can be determined. The administration frequency can be, for example, once every week, 2 weeks, 3 weeks, 4 weeks, 2 months, 3 months, 4 months, 5 months, 6 months, 1 year or 2 years. Since tracking of antibody titer may require considerable labor, it is also possible, for example, to periodically administer the phospholipid membrane preparation of the present invention to prevent infectious diseases.

When the phospholipid membrane preparation of the present invention is used as a therapeutic drug for allergy or a vaccine for the prophylaxis of infectious diseases, it characteristically suppresses the production of IgE antibody and potentiates practically sufficient production of IgG antibody. Further realization of these characteristics of the present invention results in a markedly small ratio of IgE antibody (antibody titer after secondary immunization)/IgG antibody (antibody titer after secondary immunization) (hereinafter to be abbreviated as “IgE/IgG ratio”) and sufficiently high IgG antibody titer, as compared to any of the pharmaceutical preparations conventionally used for medical practice in this field. The markedly lower IgE/IgG ratio than in a vaccine using, as an adjuvant, an aluminum hydroxide gel with many examples in practice, and the like, and further, the markedly high IgG antibody titer are very important and highly significant for a vaccine used for the treatment of allergy or the prophylaxis of infectious diseases. The markedly lower IgE/IgG ratio means a lower level of allergic conditions due to IgE antibody, by a vaccine therapy for the treatment of allergy and the prophylaxis of infectious diseases. In addition, a markedly higher IgG antibody titer further contributes to lowering of the IgE/IgG ratio by the treatment of allergy. In the vaccine therapy for the prophylaxis of infectious diseases, the IgG antibody is one of the main functions of prevention of infectious diseases, and the IgG antibody titer is preferably still higher.

Since the phospholipid membrane preparation of the present invention can achieve markedly high IgG antibody production, the infection preventive function can be achieved even when it is realized in a preparation containing an antigen at a lower concentration. A vaccine preparation containing an antigen, which is a foreign substance for living organisms, is associated with a permanent problem of avoidance of various side effects and adverse influence due to the antigen. In this respect, since the phospholipid membrane preparation of the present invention having a markedly high IgG antibody producing ability can contain an antigen at a low concentration level, it is a highly preferable technique. As used herein, by the above-mentioned “IgE/IgG ratio” is meant the ratio of titers when blood IgG antibody and IgE antibody are measured by ELISA method. It is appreciated that, even when the method of measuring IgG antibody and IgE antibody is different, the production of IgE antibody can be suppressed and practically sufficient production of IgG antibody can be increased by evaluating the immunity controlling ability using the IgE/IgG ratio based on a similar concept.

The ELISA method used in the present invention is described in the following.

[ELISA Method (IgG Antibody Detection)]

1) Coating of Plate with Antigen

An antigen is dissolved in 0.05 M carbonate buffer (pH 9.0) at a concentration of 1 mg/ml, dispensed to a 96 well assay plate at 50 μl/well and left standing at room temperature for 1 hr.

2) Blocking of Plate

Bovine serum albumin (hereinafter to be referred to as “BSA”) is dissolved in 0.2 M phosphate buffer (pH 7.2: hereinafter to be referred to as “PBS”) at a concentration of 1 mg/ml, dispensed to the plate of the above-mentioned 1) at 100 μl/well and left standing at room temperature for 1 hr.

3) Dilution and Addition of Serum Sample (Primary Antibody)

Serum of mouse immunized with aluminum hydroxide-antigen, or the phospholipid membrane preparation of the present invention is diluted in PBS containing BSA at a concentration of 1 mg/ml (hereinafter to be referred to as “PBSA”), 11 times starting from 10-fold dilution in two-fold series, dispensed to the plate of the above-mentioned 2) at 50 μl/well and left standing at room temperature for 1 hr.

4) Addition of Peroxidase Labeled Rabbit Anti-Mouse IgG Antibody Solution (Secondary Antibody)

The plate of the above-mentioned 3) is washed 3 times with PBS, a solution of peroxidase labeled rabbit anti-mouse IgG antibody in PBSA is dispensed thereto at 50 μl/well and the plate is left standing at room temperature for 1 hr.

5) Addition of Enzyme Substrate Solution

The plate of the above-mentioned 4) is washed 3 times with PBS, o-phenylenediamine dihydrochloride (0.5 mg/ml) dissolved in citrate buffer is dispensed thereto at 100 μl/well and the plate is left standing at room temperature for 15 min. to allow color development. After color development, 2 M sulfuric acid is dispensed thereto at 50 μl/well to stop the reaction.

6) Measurement Using Absorbance Meter

Absorbance at 490 nm is measured using an ELISA plate reader. The end point of color development is determined, and the average dilution fold of the sample serum at the point is taken as an ELISA titer (ELISA titer is used as a quantification value of IgG antibody).

[ELISA Method (IgE Antibody Detection)]

1) Coating of Plate with Rat Monoclonal Antibody Mouse IgE Antibody

A rat monoclonal antibody against mouse IgE antibody is adjusted to a concentration of 4 mg/ml with 0.05 M carbonate buffer (pH 9.0), dispensed to a 96 well assay plate at 100 μl/well and left standing at 37° C. for 3 hr.

2) Blocking of Plate

Bovine serum albumin (BSA) is dissolved in 0.2 M phosphate buffer (pH 7.2: PBS) at a concentration of 1 mg/ml, dispensed to the plate of the above-mentioned 1) at 100 μl/well and left standing at room temperature for 1 hr.

3) Dilution and Addition of Serum Sample (Primary Antibody)

Serum of mouse immunized with aluminum hydroxide-antigen, or the phospholipid membrane preparation of the present invention is diluted in PBS containing BSA at a concentration of 1 mg/ml (PBSA), 11 times starting from 10-fold dilution in two-fold series, dispensed to the plate of the above-mentioned 2) at 50 μl/well and left standing at room temperature for 1 hr.

4) Addition of Biotinylated Antigen

The plate of the above-mentioned 3) is washed 3 times with PBS, a solution (1 mg/ml) of biotinylated antigen solution in PBSA is dispensed thereto at 100 μl/well and the plate is left standing at room temperature for 1 hr.

5) Addition of Peroxidase Labeled Streptoavidin Solution

The plate of the above-mentioned 4) is washed 3 times with PBS, a peroxidase labeled streptoavidin solution is dispensed thereto at 100 μl/well and the plate is left standing at room temperature for 1 hr.

6) Addition of Enzyme Substrate Solution

The plate of the above-mentioned 5) is washed 3 times with PBS, o-phenylenediamine dihydrochloride (0.5 mg/ml) dissolved in citrate buffer is dispensed thereto at 100 μl/well and the plate is left standing at room temperature for 15 min. to allow color development. After color development, 2 M sulfuric acid is dispensed thereto at 50 μl/well to stop the reaction.

7) Measurement Using Absorbance Meter

Absorbance at 490 nm is measured using an ELISA plate reader. The end point of color development is determined, and the average dilution fold of the sample serum at the point is taken as an ELISA titer (ELISA titer is used as a quantification value of IgE).

### EXAMPLES

The present invention is more specifically explained in the following based on Examples and Comparative Examples, which are not to be construed as limitative.

### Example 1

(Preparation of Liposome)

1) Preparation of Lipid Mixed Powder

Didodecanoylphosphatidylcholine (0.7560 g, 1.2157 mmol), didodecanoylphosphatidylethanolamine (0.5287 g, 0.9118 mmol), cholesterol (0.8223 g, 2.1274 mmol) and didodecanoylphosphatidylserine Na salt (0.3927 g, 0.6078 mmol) were charged in an eggplant shaped flask, and a mixed solvent of chloroform/methanol/water (65/25/4, volume ratio, 50 ml) was added to allow dissolution at 40° C. Using a rotary evaporator, the solvent was evaporated under reduced pressure to give a thin lipid membrane. Furthermore, injectable distilled water (30 ml) was added and the mixture was stirred to give a homogeneous slurry. This slurry was frozen with liquid nitrogen and dried in a freeze dryer for 24 hr to give a lipid mixed powder.

2) Preparation of Liposome

Then, a buffer (0.12 mM Na₂HPO₄, 0.88 mM KH₂PO₄, 0.25M saccharose, pH 6.5, hereinafter to be abbreviated as “buffer”, 60 ml) prepared separately was placed in an eggplant shaped flask containing the above-mentioned lipid mixed powder and the mixture was stirred at 40° C. to hydrate the lipid, whereby a liposome was obtained. Then, using an extruder, the particle size of the liposome was adjusted. First, the obtained liposome was passed through a 8 μm polycarbonate filter and then sequentially passed through 5 μm, 3 μm, 1 μm, 0.65 μm, 0.4 μm and 0.2 μm filters. The average particle size of the liposome particles was 191 nm (measurement by dynamic light scattering method).

(Administration Immunity Test with OVA-Bound Liposome Suspension)

1) Preparation of Liposome Preparation

The obtained liposome (2 ml) was placed in a test tube, and 0.5 ml of ovalbumin (Sigma, reagent, hereinafter sometimes to be referred to as “OVA”) solution (12 mg/ml) was added. Then, 2.4% glutaraldehyde solution (0.5 ml) was added dropwise and gently mixed on a warm bath at 37° C. for 1 hr to immobilize the ovalbumin on the outer aqueous phase side of the liposome. Then, 2 M glycine—NaOH buffer (pH 7.2, 0.5 ml) was added and the solution was left standing overnight at 4° C. to inactivate unreacted glutaraldehyde. Furthermore, this solution was passed through a column packed with Sepharose CL-4B (Pharmacia Biotech, trademark) to fractionate the object product, whereby a liposome suspension wherein an antigen is bound to the surface of a phospholipid membrane was obtained. The phosphorus concentration of the liposome suspension was measured (phospholipid test Wako), diluted with a buffer to adjust the phosphorus concentration derived from phospholipid to 2 mM, whereby an OVA-bound liposome suspension was obtained. Using a radiolabeled OVA, the same operation as above was performed, and the amount of OVA bound when the phosphorus concentration derived from phospholipid of the liposome was 2 mM was measured and found to be 49 μg/ml.

2) Antibody Production Test

Using BALB/c Mice (Female, 8-week-old, 6 mice/group), OVA-bound liposome suspension (200 μl/injection) was intraperitoneally administered with syringe. Four weeks later, the administration was performed by a similar method for secondary immunization. Serum was taken every week from the start of the test to 6 weeks later, and the shift of the antibody titer (IgG and IgE) was measured by ELISA method.

3) Measurement of IgG Antibody by ELISA Method

Using the mouse serum obtained from the start of the immunity test to week 6, IgG antibody titer was measured by the following method.

a) Coating of Plate with Antigen

An ovalbumin was dissolved in 0.05 M carbonate buffer (pH 9.0) at a concentration of 1 mg/ml, dispensed to a 96 well assay plate at 50 μl/well and left standing at room temperature for 1 hr.

b) Blocking of Plate

Bovine serum albumin (hereinafter to be referred to as “BSA”) was dissolved in 0.2 M phosphate buffer (pH 7.2: hereinafter to be referred to as “PBS”) at a concentration of 1 mg/ml, dispensed to the plate of the above-mentioned a) at 100 μl/well and left standing at room temperature for 1 hr.

c) Dilution and Addition of Serum Sample (Primary Antibody)

Serum of mouse immunized with antigen bound liposome preparation was diluted in PBS containing BSA, at a concentration of 1 mg/ml (hereinafter to be referred to as “PBSA”), 11 times starting from 10-fold dilution in two-fold series, dispensed to the plate of the above-mentioned b) at 50 μl/well and left standing at room temperature for 1 hr.

d) Addition of Peroxidase Labeled Rabbit Anti-Mouse IgG Antibody Solution (Secondary Antibody)

The plate of the above-mentioned c) was washed 3 times with PBS, a solution of peroxidase labeled rabbit anti-mouse IgG antibody in PBSA is dispensed thereto at 50 μl/well and the plate was left standing at room temperature for 1 hr.

e) Addition of Enzyme Substrate Solution

The plate of the above-mentioned d) was washed 3 times with PBS and dissolved in citrate buffer, o-phenylenediamine dihydrochloride (0.5 mg/ml) was dispensed thereto at 100 μl/well and the plate was left standing at room temperature for 15 min to allow color development. After color development, 2 M sulfuric acid was dispensed thereto at 50 μl/well to stop the reaction.

f) Measurement Using Absorbance Meter

Absorbance at 490 nm was measured using an ELISA plate reader. The end point of color development is determined, and the dilution fold of the sample serum at the point was taken as an ELISA titer (ELISA titer was used as a quantification value of IgG antibody).

4) Measurement of IgE Antibody by ELISA Method

Using the mouse serum obtained from the start of the immunity test to week 6, IgE antibody titer was measured by the following method.

a) Coating of Plate with Rat Monoclonal Antibody of Mouse IgE Antibody

A rat monoclonal antibody against mouse IgE antibody was adjusted to a concentration of 4 μg/ml with 0.05 M carbonate buffer (pH 9.0), dispensed to a 96 well assay plate at 100 μl/well and left standing at 37° C. for 3 hr.

b) Blocking of Plate

Bovine serum albumin (BSA) was dissolved in 0.2 M phosphate buffer (pH 7.2: PBS) at a concentration of 1 mg/ml, dispensed to the plate of the above-mentioned a) at 100 μl/well and left standing at room temperature for 1 hr.

c) Dilution and Addition of Serum Sample (Primary Antibody)

Serum of mouse immunized with aluminum hydroxide-antigen, or the liposome preparation of the present invention was diluted in PBS containing BSA, at a concentration of 1 mg/ml (PBSA), 11 times starting from 10-fold dilution in two-fold series, dispensed to the plate of the above-mentioned b) at 50 μl/well and left standing at room temperature for 1 hr.

d) Addition of Biotinylated Antigen

The plate of the above-mentioned c) was washed 3 times with PBS, a solution (1 μg/ml) of biotinylated antigen solution in PBSA was dispensed thereto at 100 μl/well and the plate was left standing at room temperature for 1 hr.

e) Addition of Peroxidase Labeled Streptoavidin Solution

The plate of the above-mentioned d) was washed 3 times with PBS, a peroxidase labeled streptoavidin solution was dispensed thereto at 100 μl/well and the plate was left standing at room temperature for 1 hr.

f) Addition of Enzyme Substrate Solution

The plate of the above-mentioned e) was washed 3 times with PBS, o-phenylenediamine dihydrochloride (0.5 mg/ml) dissolved in citrate buffer was dispensed thereto at 100 μl/well and the plate was left standing at room temperature for 15 min to allow color development. After color development, 2 M sulfuric acid was dispensed thereto at 50 μl/well to stop the reaction.

g) Measurement Using Absorbance Meter

Absorbance at 490 nm was measured using an ELISA plate reader. The end point of color development was determined, and the average dilution fold of the sample serum at the point was taken as an ELISA titer (ELISA titer was used as a quantification value of IgE antibody).

5) Results of Antibody Titer;

The antibody titers of IgG and IgE at week 6, and the IgE/IgG ratio calculated from the antibody titers thereof are shown in Table 3.

### Example 8

(Preparation of Liposome)

1) Synthesis of Reactive Phospholipid Consisting of Terminal Modified Phosphatidyl-ethanolamine (succinimidyl-didecanoylphosphatidylethanolamine)

Didecanoylphosphatidylethanolamine (2 g) and triethylamine (180 μl) were dissolved in and added to chloroform (50 ml), and the mixture was placed in a 300 ml 4-mouthed flask. The inside of the flask was stirred with a magnet stirrer at room temperature and a solution prepared separately, wherein disuccinimidylsuccinate (3 g), which is a bifunctional reactive compound, was dissolved in chloroform (80 ml), was added dropwise over 4 hr according to a conventional method to allow reaction of an amino group of didecanoylphosphatidylethanolamine with one terminal of disuccinimidylsuccinate. This crude reaction mixture was transferred to an eggplant shaped flask, and the solvent was evaporated with an evaporator. Then, a small amount of chloroform sufficient to dissolve the crude reaction product was added to this flask to give a high concentration crude reaction product solution, which was then subjected to column chromatography according to a conventional method using silica gel equilibrated with chloroform/methanol/water (65/25/1, volume ratio). Only a fraction wherein one terminal of disuccinimidylsuccinate is bound to an amino group of the object didecanoylphosphatidylethanolamine was recovered. The solvent was evaporated to give succinimidyl-didecanoylphosphatidylethanolamine, which is the object reactive phospholipid.

2) Preparation of Lipid Mixed Powder

Didodecanoylphosphatidylcholine (0.0337 g, 0.0541 mmol), succinimidyl-didecanoylphosphatidylethanolamine (0.2165 g, 0.2705 mmol) prepared in the above-mentioned 1), cholesterol (0.5021 g, 1.2986 mmol) and didodecanoylphosphatidylserine Na salt (1.7477 g, 2.706 mmol) were charged in an eggplant shaped flask, and a mixed solvent of chloroform/methanol/water (65/25/4, volume ratio, 50 ml) was added to allow dissolution at 40° C. Using a rotary evaporator, the solvent was evaporated under reduced pressure to give a thin lipid membrane. Furthermore, injectable distilled water (30 ml) was added and the mixture was stirred to give a homogeneous slurry. This slurry was frozen with liquid nitrogen and dried in a freeze dryer for 24 hr to give a lipid mixed powder.

3) Preparation of Liposome

By a similar method as in Example 1, “2) Preparation of liposome”, a liposome was prepared. The average particle size of the liposome particles was 181 nm (measurement by dynamic light scattering method).

(Administration Immunity Test with OVA-Bound Liposome Suspension)

1) The obtained liposome (1.5 ml) was placed in a test tube, and 3 ml of ovalbumin (Sigma, reagent, hereinafter sometimes to be referred to as “OVA”) solution (1.25 mM, buffer solution) prepared separately was added. The mixture was gently mixed at 5° C. for 48 hr to allow reaction. This reaction mixture was subjected to gel filtration according to a conventional method using Sepharose CL-4B equilibrated with a buffer. Since liposome fraction is clouded, the object fraction can be easily confirmed. It is also possible to confirm using a UV detector and the like. The phosphorus concentration of the obtained liposome suspension was measured (phospholipid test Wako), diluted with a buffer to adjust the phosphorus concentration derived from phospholipid to 2 mM, whereby an OVA-bound liposome suspension was obtained. The amount of OVA bound when the phosphorus concentration derived from phospholipid of the liposome was 2 mM was 38 μg/ml.

2) By a similar method as in Example 1, antibody production test, measurement of IgG antibody by ELISA method and measurement of IgE antibody by ELISA method were performed. The antibody titers of IgG and IgE at week 6, and the IgE/IgG ratio calculated from the antibody titers thereof are shown in Table 3.

### Example 9

(Preparation of Liposome)

1) Synthesis of Reactive Phospholipid Consisting of Terminal Modified Phosphatidylethanolamine (maleimide-didodecanoylphosphatidylethanolamine)

In the same manner as in Example 8 except that disuccinimidylsuccinate in “1) synthesis of reactive phospholipid consisting of terminal modified phosphatidylethanolamine” was changed to N-succinimidyl-4-(p-maleimidephenyl)propionate, and using the same mol numbers of didodecanoylphosphatidylethanolamine, triethylamine and bifunctional reactive compound used and similarly performing the subsequent steps, maleimide-didodecanoylphosphatidylethanolamine, which was the object reactive phospholipid, was obtained.

2) Preparation of Lipid Mixed Powder

Didecanoylphosphatidylcholine (1.0425 g, 1.8428 mmol), maleimide-didodecanoylphosphatidylethanolamine (0.2375 g, 0.3071 mmol) prepared in the above-mentioned 1), cholesterol (0.8313 g, 2.1499 mmol) and didecanoylphosphatidylglycerol Na salt (0.3888 g, 0.6143 mmol) were charged in an eggplant shaped flask, and a mixed solvent of chloroform/methanol/water (65/25/4, volume ratio, 50 ml) was added to allow dissolution at 40° C. Using a rotary evaporator, the solvent was evaporated under reduced pressure to give a thin lipid membrane. Furthermore, injectable distilled water (30 ml) was added and the mixture was stirred to give a homogeneous slurry. This slurry was frozen with liquid nitrogen and dried in a freeze dryer for 24 hr to give a lipid mixed powder.

3) Preparation of Liposome

By a similar method as in Example 1, “2) Preparation of liposome”, a liposome was prepared. The average particle size of the liposome particles was 165 nm (measurement by dynamic light scattering method).

(Administration Immunity Test with OVA-Bound Liposome Suspension)

1) The obtained liposome (1.5 ml) was placed in a test tube, and 3 ml of ovalbumin (Sigma, reagent, hereinafter sometimes to be referred to as “OVA”) solution (1.25 mM, buffer solution) prepared separately was added. The mixture was gently stirred at 5° C. for 48 hr to allow reaction. This reaction mixture was subjected to gel filtration according to a conventional method using Sepharose CL-4B equilibrated with a buffer. Since liposome fraction is clouded, the object fraction can be easily confirmed. It is also possible to confirm using a UV detector and the like. The phosphorus concentration of the obtained liposome suspension was measured (phospholipid test Wako), diluted with a buffer to adjust the phosphorus concentration derived from phospholipid to 2 mM, whereby an OVA-bound liposome suspension was obtained. The amount of OVA bound when the phosphorus concentration derived from phospholipid of the liposome was 2 mM was 40 μg/ml.

2) By a similar method as in Example 1, antibody production test, measurement of IgG antibody by ELISA method and measurement of IgE antibody by ELISA method were performed. The antibody titers of IgG and IgE at week 6, and the IgE/IgG ratio calculated from the antibody titers thereof are shown in Table 3.

### Examples 2-7 and 10

(Preparation of Liposome, and Administration Immunity Test with OVA-Bound Liposome Suspension)

1) According to the respective molar ratios of phospholipid and cholesterol added as shown in Table 1, and in the same manner as in Example 1, the liposomes of Examples 2-7 and 10 were obtained. Using the liposomes obtained in Examples 2-7 and 10, and in the same manner as in Example 1, respective suspensions of OVA-bound liposome were prepared.

The particle size (nm) of the liposomes obtained in Examples 2-7 and 10 were 188, 171, 149, 151, 154, 155 and 147, respectively (measurement by dynamic light scattering method).

In addition, the amounts (μg/ml) of OVA bound when the phosphorus concentration derived from phospholipid of the liposomes obtained in Examples 2-7 and 10 was 2 mM were 44, 46, 55, 57, 53, 51 and 51, respectively.

2) With regard to the suspensions of OVA-bound liposomes obtained in Examples 2-7 and 10, the antibody production test, measurement of IgG antibody by ELISA method and measurement of IgE antibody by ELISA method were performed by the same methods as in Example 1. The antibody titers of IgG and IgE at week 6, and the IgE/IgG ratio calculated from the antibody titers thereof are shown in Table 3.

TABLE 1

| | | Number of | | | | | | | | | | |
|---|---|---|---|---|---|---|---|---|---|---|---|---|
| Constituent | | carbon of | | | | | | | | | | |
| components | | acyl group | Ex. 1 | Ex. 2 | Ex. 3 | Ex. 4 | Ex. 5 | Ex. 6 | Ex. 7 | Ex. 8 | Ex. 9 | Ex. 10 |
| Neutral | PC | 10 | — | — | — | 12.50 | 40.00 | 25.00 | 20.00 | — | 37.50 | 44.00 |
| phospholipid | | 11 | — | — | 25.00 | — | — | — | — | — | — | — |
| | | 12 | 25.00 | — | — | — | — | — | — | 1.25 | — | — |
| Reactive | PE | 10 | — | — | — | 18.75 | 30.00 | 18.75 | 15.00 | — | — | 33.00 |
| phospholipid | | 12 | 18.75 | 18.75 | 18.75 | — | — | — | — | — | — | — |
| | SI-PE | 10 | — | — | — | — | — | — | — | 6.25 | — | — |
| | MI-PE | 12 | — | — | — | — | — | — | — | — | 6.25 | — |
| Cholesterol | | | 43.75 | 43.75 | 43.75 | 43.75 | 10.00 | 43.75 | 55.00 | 30.00 | 43.75 | 1.00 |
| Acidic | PS | 10 | — | — | — | 25.00 | 20.00 | — | 10.00 | — | — | — |
| phospholipid | | 12 | 12.50 | 37.50 | 12.50 | — | — | — | — | 62.50 | — | — |
| | PG | 10 | — | — | — | — | — | 12.50 | — | — | 12.50 | 22.00 |

PC: diacylphosphatidylcholine,
PS: diacylphosphatidylserine,
PG: diacylphosphatidylglycerol,
PE: diacylphosphatidylethanolamine,
SI-PE: succinimidyl-diacylphosphatidylethanolamine,
MI-PE: maleimide-diacylphosphatidylethanolamine

### Comparative Example 1

1) Preparation of Aluminum Hydroxide Gel Suspension

OVA was dissolved in a buffer (1.2 mM Na₂HPO₄, 8.8 mM KH₂PO₄, pH 6.5) prepared separated to achieve 500 mg/ml, and this OVA solution (1 ml) was added to an aluminum hydroxide gel (500 μg/ml) suspension (9 ml) prepared according to a conventional method to give an OVA-aluminum hydroxide gel suspension. The OVA concentration of this OVA-aluminum hydroxide gel suspension was 50 μg/ml.

(Administration Immunity Test of Aluminum Hydroxide Gel Suspension)

2) Antibody Production Test

In the same manner as in Example 1 except that the OVA-aluminum hydroxide gel suspension prepared in the above-mentioned 1) was used instead of the liposome preparation, an antibody production test was performed. Serum was taken every week from the start of the test to 6 weeks later, and the shift of the antibody titer (IgG and IgE) was measured by ELISA method.

3) Measurement of IgG antibody by ELISA Method

Using the mouse sera obtained in the above-mentioned 2), which were taken from the start of the immunity test to week 6 and in the same manner as in Example 1, the IgG antibody titer was measured. The results are shown in Table 3.

4) Measurement of IgE Antibody by ELISA Method

Using the mouse sera obtained in the above-mentioned 3), which were taken from the start of the immunity test to week 6 and in the same manner as in Example 1, the IgE antibody titer was measured. The results are shown in Table 3.

5) Results of Antibody Titer

The antibody titers of IgG and IgE at week 6, and the IgE/IgG ratio calculated from the antibody titers thereof are shown in Table 3.

TABLE 2

| | | Number of | | | | | | | | |
|---|---|---|---|---|---|---|---|---|---|---|
| Constituent | | carbon of acyl | Comp. | Comp. | Comp. | Comp. | Comp. | Comp. | Comp. | Comp. |
| components | | group | Ex. 1 | Ex. 2 | Ex. 3 | Ex. 4 | Ex. 5 | Ex. 6 | Ex. 7 | Ex. 8 |
| Neutral | PC | 14 | — | — | — | — | 25.00 | — | — | — |
| phospholipid | | 16 | — | — | 25.00 | 25.00 | — | 37.50 | 37.50 | 44.44 |
| | | 18 | — | 25.00 | — | — | — | — | — | — |
| Reactive | PE | 14 | — | — | — | — | 18.75 | — | — | — |
| phospholipid | | 16 | — | — | 18.75 | 18.75 | — | — | — | 33.33 |
| | | 18 | | 18.75 | — | — | — | — | — | — |
| | SI-PE | 16 | — | — | — | — | — | 6.25 | — | — |
| | MI-PE | 18 | — | — | — | — | — | — | 6.25 | — |
| Cholesterol | | | — | 43.75 | 43.75 | 43.75 | 44.75 | 43.75 | 43.75 | 0 |
| Acidic | — | 16 | — | — | 12.5 | — | — | — | — | — |
| phospholipid | | 18 | — | 12.5 | — | — | — | — | — | — |
| | PG | 14 | — | — | — | 12.5 | 12.5 | — | 12.5 | 22.22 |
| | | 16 | — | — | — | — | — | 12.5 | — | — |

| | | | | | | | | |
|---|---|---|---|---|---|---|---|---|
| Aluminum hydroxide gel adjuvant | 100 | — | — | — | — | — | — | — |

PC: diacylphosphatidylcholine,
PS: diacylphosphatidylserine,
PG: diacylphosphatidylglycerol,
PE: diacylphosphatidylethanolamine,
SI-PE: succinimidyl-diacylphosphatidylethanolamine,
MI-PE: maleimide-diacylphosphatidylethanolamine

### Comparative Examples 2-5 and 8

(Preparation of Liposomes and Administration Immunity Test of OVA-Bound Liposome Suspensions)

1) According to the respective combination molar ratios of phospholipid and cholesterol added as shown in Table 2, and in the same manner as in Example 1, the liposomes of Comparative Examples 2-5 and 8 were obtained. Using the liposomes of Comparative Examples 2-5 and 8, and in the same manner as in Example 1, respective suspensions of OVA-bound liposome were prepared.

The particle size (nm) of the liposomes obtained in Comparative Examples 2-5 and 8 were 263, 248, 242, 218 and 251, respectively (measurement by dynamic light scattering method).

In addition, the amounts (μg/ml) of OVA bound when the phosphorus concentration derived from phospholipid of the liposomes obtained in Comparative Examples 2-5 and 8 was 2 mM were 45, 50, 53, 56 and 46, respectively.

2) With regard to the suspensions of OVA-bound liposomes obtained in Comparative Examples 2-5 and 8, the antibody production test, measurement of IgG antibody by ELISA method and measurement of IgE antibody by ELISA method were performed by the same methods as in Example 1. The antibody titers of IgG and IgE at week 6, and the IgE/IgG ratio calculated from the antibody titers thereof are shown in Table 3.

### Comparative Examples 6 and 7

(Preparation of Liposomes and Administration Immunity Test of OVA-Bound Liposome Suspensions)

1) According to the respective combination molar ratios of phospholipid and cholesterol added as shown in Table 2, and in the same manner as in Examples 8 and 9, the liposomes of Comparative Examples 6 and 7 were obtained. Using the liposomes obtained in Comparative Examples 6 and 7, and in the same manner as in Examples 8 and 9, respective suspensions of OVA-bound liposome were prepared.

The particle size (nm) of the liposomes obtained in Comparative Examples 6 and 7 were 252 and 255, respectively.

In addition, the amounts (μg/ml) of OVA bound when the phosphorus concentration derived from phospholipid of the liposomes obtained in Comparative Examples 6 and 7 was 2 mM were 39 and 38, respectively.

2) With regard to the suspensions of OVA-bound liposomes obtained in Comparative Examples 6 and 7, the antibody production test, measurement of IgG antibody by ELISA method and measurement of IgE antibody by ELISA method were performed by the same methods as in Example 1. The antibody titers of IgG and IgE at week 6, and the IgE/IgG ratio calculated from the antibody titers thereof are shown in Table 3.

TABLE 3

| | | Evaluation | | | Evalua- | |
|---|---|---|---|---|---|---|
| | IgG | of IgG | | | tion of | Overall |
| Evaluation | antibody | antibody | | IgE/IgG | IgE/IgG | evalua- |
| item | titer | titer | IgE | ratio | ratio | tion |
| Comp. Ex. 1 | 820 | ⊙ | 11.30 | 0.0138 | X | X |
| Comp. Ex. 2 | 45 | X | 0.10 | 0.0022 | ◯ | X |
| Comp. Ex. 3 | 95 | X | 0.00 | 0.0000 | ⊙ | X |
| Comp. Ex. 4 | 78 | X | 0.00 | 0.0000 | ⊙ | X |
| Comp. Ex. 5 | 162 | Δ | 0.05 | 0.0003 | ⊙ | X |
| Comp. Ex. 6 | 41 | X | 0.00 | 0.0000 | ⊙ | X |
| Comp. Ex. 7 | 52 | X | 0.20 | 0.0038 | ◯ | X |
| Comp. Ex. 8 | 154 | Δ | 1.80 | 0.0117 | X | X |
| Ex. 1 | 922 | ⊙ | 0.10 | 0.0001 | ⊙ | ⊙ |
| Ex. 2 | 1154 | ⊙ | 0.00 | 0.0000 | ⊙ | ⊙ |
| Ex. 3 | 1487 | ⊙ | 0.00 | 0.0000 | ⊙ | ⊙ |
| Ex. 4 | 2315 | ⊙ | 0.15 | 0.0001 | ⊙ | ⊙ |
| Ex. 5 | 2109 | ⊙ | 0.24 | 0.0001 | ⊙ | ⊙ |
| Ex. 6 | 2296 | ⊙ | 0.10 | 0.0000 | ⊙ | ⊙ |
| Ex. 7 | 1254 | ⊙ | 0.00 | 0.0000 | ⊙ | ⊙ |
| Ex. 8 | 2003 | ⊙ | 0.00 | 0.0000 | ⊙ | ⊙ |
| Ex. 9 | 2236 | ⊙ | 0.10 | 0.0000 | ⊙ | ⊙ |
| Ex. 10 | 2310 | ⊙ | 0.61 | 0.0003 | ⊙ | ⊙ |

In Table 3, respective evaluation criteria of the immunity test results are as follows.

1) For IgG antibody, Δ: not less than 150 and less than 500, ◯: not less than 500 and less than 800, ⊚: not less than 800, x: less than 150.

2) For IgE/IgG ratio, ⊚; not more than 0.001, ◯; more than 0.001 and not more than 0.005, Δ; more than 0.005 and not more than 0.010, x; more than 0.010.

3) Overall Evaluation

In the IgG antibody titer and IgE/IgG ratio, the overall evaluation of the one having not less than one “x” or “Δ” was “x”. Other than this, the overall evaluation was “⊚”.

It was confirmed that the liposome preparations of Examples 1-10 showed practically sufficiently enhanced production of IgG antibody, based on the IgG antibody titer, and suppression of IgE antibody production, based on the IgE/IgG ratio. It was also confirmed that these properties were simultaneously satisfied.

From the results, it was confirmed that the above-mentioned liposome preparations had an immune response controlling function. Consequently, the above-mentioned liposome preparations are preferable as a phospholipid membrane preparation to be used as a vaccine that does not easily cause allergic conditions or used for the treatment of allergy.

This application is based on application No. 2003-360047 filed in Japan, the contents of which are incorporated hereinto by reference.

1. A phospholipid membrane preparation comprising a phospholipid membrane comprising a phospholipid having an acyl group or a hydrocarbon group having 10 to 12 carbon atoms and a stabilizer of a phospholipid membrane, wherein an antigen or an allergen is bound to the surface of the membrane.

2. The phospholipid membrane preparation of claim 1, wherein said phospholipid is at least one kind selected from diacylphosphatidylserine, diacylphosphatidylglycerol, diacylphosphatidic acid, diacylphosphatidylinositol, diacylphosphatidylcholine, diacylphosphatidylethanolamine, succinimidyl-diacylphosphatidylethanolamine and maleimide-diacylphosphatidylethanolamine.

3. The phospholipid membrane preparation of claim 1, wherein said phospholipid comprises at least one selected from diacylphosphatidylserine, diacylphosphatidylglycerol and diacylphosphatidic acid.

4. The phospholipid membrane preparation of claim 1, which comprises a lipid wherein an antigen or an allergen is bound by an ionic bond or a covalent bond.

5. The phospholipid membrane preparation of claim 1, wherein said stabilizer is cholesterol.

6. The phospholipid membrane preparation of claim 1, wherein the phospholipid membrane preparation is a liposome preparation.

7. A liposome preparation comprising 1-99.6 mol % of an acidic phospholipid comprising an acyl group or a hydrocarbon group having 10 to 12 carbon atoms, 0.2-75 mol % of a stabilizer of a phospholipid membrane and 0.2-80 mol % of a lipid comprising an acyl group or a hydrocarbon group having 10 to 12 carbon atoms, which is bound to an antigen or an allergen.

8. A liposome preparation comprising 1-99.5 mol % of an acidic phospholipid comprising an acyl group or a hydrocarbon group having 10 to 12 carbon atoms, 0.2-75 mol % of a stabilizer of a phospholipid membrane, 0.2-80 mol % of a lipid comprising an acyl group or a hydrocarbon group having 10 to 12 carbon atoms, which is bound to an antigen or an allergen and 0.1-70 mol % of a neutral phospholipid having an acyl group or a hydrocarbon group having 10 to 12 carbon atoms.

9. A phospholipid membrane comprising a phospholipid having an acyl group or a hydrocarbon group having 10 to 12 carbon atoms, a reactive lipid and a stabilizer of a phospholipid membrane.

10. A phospholipid membrane comprising 1-99.6 mol % of an acidic phospholipid comprising an acyl group or a hydrocarbon group having 10 to 12 carbon atoms, 0.2-75 mol % of a stabilizer of a phospholipid-membrane and 0.2-80 mol % of a reactive lipid having an acyl group or a hydrocarbon group having 10 to 12 carbon atoms.

11. A phospholipid membrane comprising 1-99.5 mol % of an acidic phospholipid comprising an acyl group or a hydrocarbon group having 10 to 12 carbon atoms, 0.2-75 mol % of a stabilizer of a phospholipid membrane, 0.2-80 mol % of a reactive lipid having an acyl group or a hydrocarbon group having 10 to 12 carbon atoms and 0.1-70 mol % of a neutral phospholipid having an acyl group or a hydrocarbon group having 10 to 12 carbon atoms.

12. The phospholipid membrane preparation of claim 2, wherein said stabilizer is cholesterol.

13. The phospholipid membrane preparation of claim 3, wherein said stabilizer is cholesterol.

14. The phospholipid membrane preparation of claim 4, wherein said stabilizer is cholesterol.

15. The phospholipid membrane preparation of claim 2, wherein the phospholipid membrane preparation is a liposome preparation.

16. The phospholipid membrane preparation of claim 3, wherein the phospholipid membrane preparation is a liposome preparation.

17. The phospholipid membrane preparation of claim 4, wherein the phospholipid membrane preparation is a liposome preparation.

18. The phospholipid membrane preparation of claim 5, wherein the phospholipid membrane preparation is a liposome preparation.

19. The phospholipid membrane preparation of claim 12, wherein the phospholipid membrane preparation is a liposome preparation.

20. The phospholipid membrane preparation of claim 13, wherein the phospholipid membrane preparation is a liposome preparation.

21. The phospholipid membrane preparation of claim 14, wherein the phospholipid membrane preparation is a liposome preparation.
